(19) United States
(12) Patent Application Publication
(10) Pub. No.: US 2025/0079021 A1
(43) Pub. Date: Mar. 6, 2025

(54) SYNCHRONIZED 3D MODEL CLIPPING SYSTEM AND METHOD

(71) Applicant: Surgical Theater, Inc., Beachwood, OH (US)

(72) Inventors: MORDECHAI AVISAR, HIGHLAND HEIGHTS, OH (US); Alon Yakob Geri, Orange Village, OH (US); Robert Louis, Newport Beach, CA (US); DAVID VIZMEG, PENINSULA, OH (US)

(73) Assignee: SURGICAL THEATER, INC., Beachwood, OH (US)

(21) Appl. No.: 18/736,990

(22) Filed: Jun. 7, 2024

Related U.S. Application Data
(60) Provisional application No. 63/471,608, filed on Jun. 7, 2023.

(51) Int. Cl. G16H 50/50; G16H 20/40

(52) U.S. Cl. CPC G16H 50/50; G16H 20/40

(57) ABSTRACT

A method, including the steps of a modeling computer: receiving data indicative of a frame of reference with respect to a vector; generating data indicative of first parameters, based on the frame of reference, for clipping a 3D model of a patient anatomy along the vector; clipping the 3D model based on the first parameters, thereby generating a first clipped portion; communicating the first clipped portion to a display; receiving data indicative of movement within the frame of reference; automatically regenerating second parameters, based on and in sync with the data indicative of the movement, for clipping the 3D model; clipping the 3D model based on the second parameters, thereby generating a second clipped portion; and communicating the second clipped portion to the display.

### CROSS-REFERENCES TO RELATED APPLICATIONS

This application claims the benefit of U.S. Provisional Patent Application Ser. No. 63/471,608 filed on Jun. 7, 2023, incorporated herein by reference.

### BACKGROUND

Surgical procedures may often be complex and time sensitive and vary in scope from one patient to another. For example, in the case of an aneurysm repair, the point of repair may vary in terms or procedural requirements depending on the exact location, size, and so on. Therefore, the efficiency of the procedure is highly critical and detailed planning based on the patient specific local geometry and physical properties of the area on which surgery is being performed is fundamental. To achieve a new level of pre-surgery preparation, 3D CT and MRI images are being increasingly utilized. However, those images offer only minor benefits, standing alone, for surgery rehearsal. Moreover, existing techniques for studying a patient's specific anatomy prior to or during surgery may be invasive to the patient.

### SUMMARY

In one example, a method includes the steps of a modeling computer: receiving data indicative of a frame of reference with respect to a vector; generating data indicative of first parameters, based on the frame of reference, for clipping a 3D model of a patient anatomy along the vector; clipping the 3D model based on the first parameters, thereby generating a first clipped portion; communicating the first clipped portion to a display; receiving data indicative of movement within the frame of reference; automatically regenerating second parameters, based on and in sync with the data indicative of the movement, for clipping the 3D model; clipping the 3D model based on the second parameters, thereby generating a second clipped portion; and communicating the second clipped portion to the display.

In another example, a synchronized clipping modeling computer includes: a first module for receiving data indicative of a frame of reference with respect to a vector; a second module for generating data indicative of first parameters, based on the frame of reference, for clipping a 3D model of a patient anatomy along the vector; a third module for clipping the 3D model based on the first parameters, thereby generating a first clipped portion; and a fourth module for communicating the first clipped portion to a display.

In another example, a system includes: a first module for receiving data indicative of a frame of reference with respect to a vector; a second module for generating data indicative of first parameters, based on the frame of reference, for clipping a 3D model of a patient anatomy along the vector; a third module for clipping the 3D model based on the first parameters, thereby generating a first clipped portion; a fourth module for communicating the first clipped portion to a display; and a fifth module for receiving data indicative of movement within the frame of reference; wherein the second module is further configured for automatically regenerating second parameters, based on and in sync with the data indicative of the movement, for clipping the 3D model; wherein the third module is further configured for clipping the 3D model based on the second parameters, thereby generating a second clipped portion; and wherein the fourth module is further configured for communicating the second clipped portion to the display.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example synchronized 3D model clipping system.

FIG. 2 illustrates an example display of 3D model clipping.

FIG. 3 illustrates an example display of 3D model clipping.

FIG. 4A illustrates an example display of 3D model clipping.

FIG. 4B illustrates an example display of 3D model clipping.

FIG. 5A illustrates an example display of 3D model clipping.

FIG. 5B illustrates an example display of 3D model clipping.

FIG. 6A illustrates an example display of 3D model clipping.

FIG. 6B illustrates an example display of 3D model clipping.

FIG. 7 illustrates an example method for synchronized 3D model clipping.

FIG. 8 illustrates an example computer implementing the example synchronized 3D model clipping computer of FIG. 1.

### DETAILED DESCRIPTION

The following acronyms and definitions will aid in understanding the detailed description:

AR—Augmented Reality—A live view of a physical, real-world environment whose elements have been enhanced by computer generated sensory elements such as sound, video, or graphics.

VR—Virtual Reality—A 3Dimensional computer generated environment which can be explored and interacted with by a person in varying degrees.

HMD—Head Mounted Display refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Avatar—An avatar represents a user inside the virtual environment.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

A surgery rehearsal and preparation tool previously described in U.S. Pat. No. 8,311,791, incorporated in this application by reference, has been developed to convert static CT and MRI medical images into dynamic and interactive multi-dimensional full spherical virtual reality, six (6) degrees of freedom models (“MD6DM”) based on a prebuilt SNAP model that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional two-dimensional patient medical scans, that gives spherical virtual reality 6 degrees of freedom (i.e. linear; x, y, z, and angular, yaw, pitch, roll) in the entire volumetric spherical virtual reality model.

The MD6DM is rendered in real time by an image generator using a SNAP model built from the patient's own data set of medical images including CT, MRI, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved and can be appreciated using the MD6DM.

The algorithm of the MD6DM rendered by the image generator takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD6DM reverts it to a 3D model by representing a 360° view of each of those points from both the inside and outside.

Described herein is a synchronized 3D model clipping system, leveraging an image generator and a MD6DM model. In particular, the synchronized 3D model clipping system enables clipping into a view of a 3D model such that the clipping is synchronized with both reference movement and navigation in order to provide increased flexibility and control of the sliced or clipped view of the 3D model, to increase depth perception, and to also hide and reveal structures of the 3D model depending on a reference point or a current position and focal point of view.

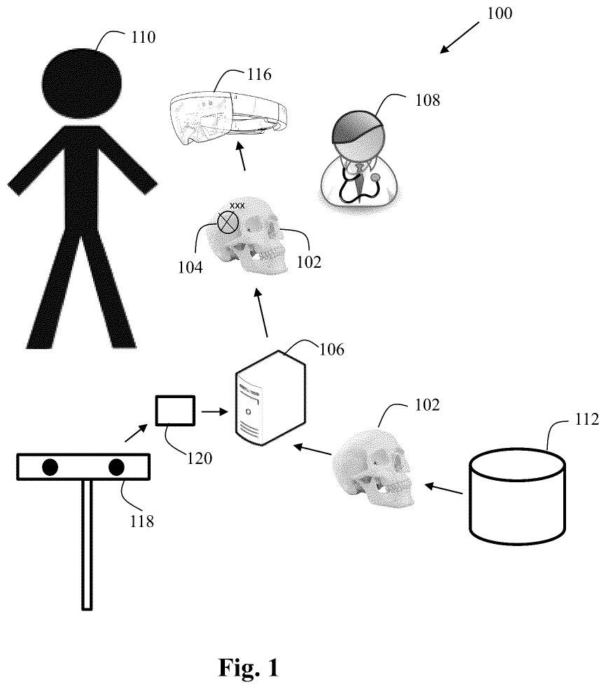

Three-dimensional models are commonly clipped, meaning that the 3D model is sliced on a flat plane so that the inside of the 3D model can be viewed from a specific perspective while the remaining portion of the model behind the plane (or closer to the user) is temporarily removed, as illustrated in FIG. 1. This allows a user to better focus on a specific portion of the 3D model. Current systems allow for clipping into the model as the user's reference point moves in and out of the model along a single clipping vector that defines the plane. However, such clipping along a single vector limits the user's ability to visualize and explore the 3D model. The 3D model clipping system described herein enables clipping into a 3D model with more flexibility and control and along a vector that can be adjusted, customized, and synchronized along different perspectives.

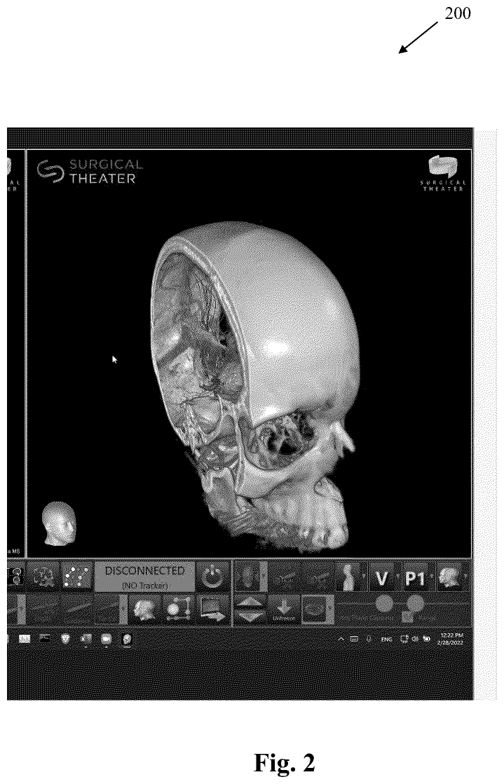

FIG. 1 illustrates a system 100 for enabling clipping into a 3D virtual model 102. In particular, the system includes a surgical planning and modeling computer (“Modeling Computer”) 106 that enables a user 108, such as a physician, to create, or retrieve from a virtual model database 112, a 3D virtual model 102, such as a SNAP model. Once the virtual model 102 retrieved, the user 108 may use the modeling computer 106 clip into the virtual model 102 in order to view a sliced or clipped portion 104 of the virtual model 102, as illustrated in an example clipped portion 200 in FIG. 2.

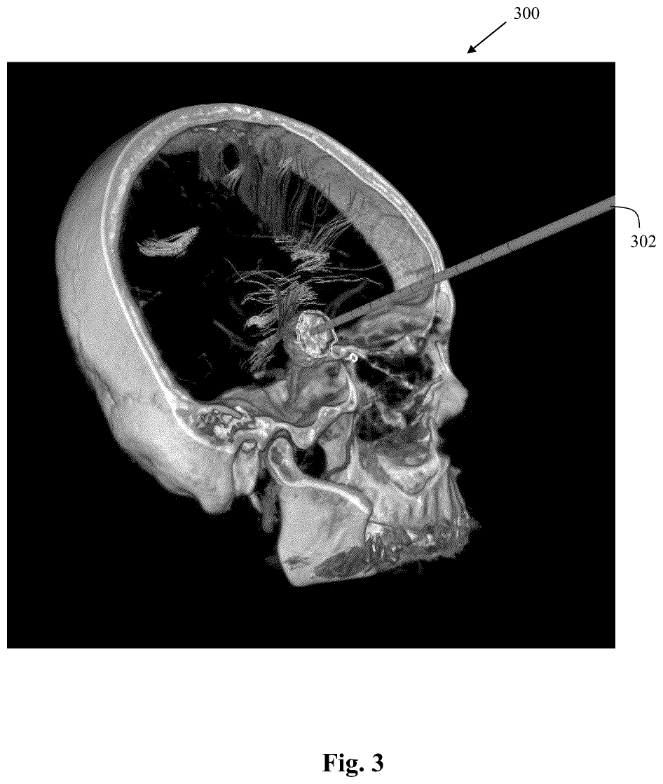

The modeling computer 106 performs a clipping of a 3D virtual model 102 by chopping or slicing the 3D model 102 along a flat plain on a focal point perpendicular to a vector and eliminating from view everything closer or in front of the flat plain on focal point, as illustrated in the example clipped portion 300 in FIG. 3. In the example illustrated, a vector is defined by a simulated probe 302. However, a vector may similarly be defined by, for example, by a microscope, or other suitable medical instruments, tools or objects capable of defining a position, angle, and reference tip or focal point. In one example, the vector may be defined by a position and angle of view of a user 108. In such an example, the modeling computer 106 is capable of receiving tracking coordinates from a device on the user 108 and to process the coordinates in order to generate a vector for clipping the 3D model.

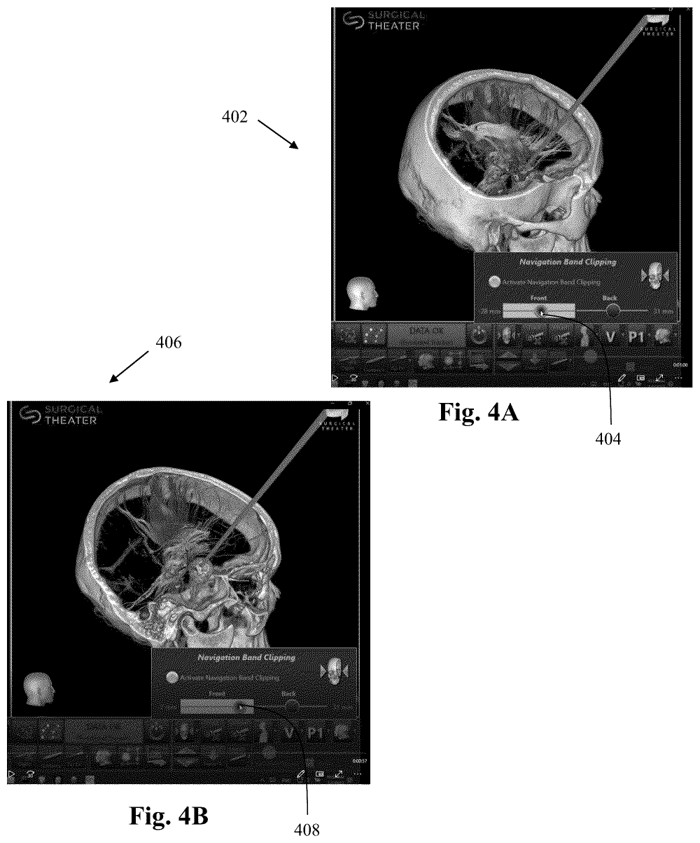

In one example, as illustrated in the clipped portions 402 and 406 in FIGS. 4a and 4b, respectively, the modeling computer 106 enables range clipping or adjusting the thickness or the range of the slice of the clipped portion 104. In other words, in addition to clipping the 3D virtual model 102 in the front side, a flat plane is also defined on the back side, at a defined distance or depth relative to the plane defined on the front side. By clipping both the front and back side, this creates a parallax effect in order to provide improved depth perception to a user 108. In the examples illustrated, the modeling computer 106 provides a slide in order for the user 108 to select the depth or range of the clipped slice. As illustrated, the user 108 may select a 28 mm range 404 using a slider for the front side in a first example, and then adjust the slider to a 7 mm range 408 for the front side in a second example. This means that the plane defining the clipping on the front side will be positioned 28 mm and 7 mm, respectively, relative to a focal point defined by a tip of a vector. Although the back side ranges in both of the illustrated examples remain constant, the user 108 may similarly adjust the range of the back side, and accordingly position the back side plane.

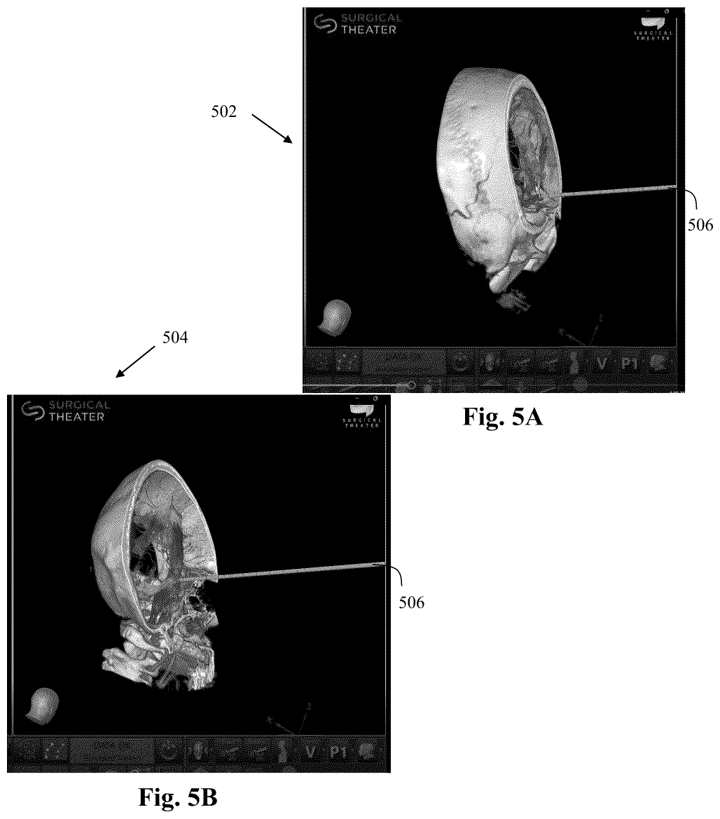

In one example, as illustrated in FIGS. 5a and 5b, the modeling computer 106 may enable range clipping and the desired depth of the clipped portion 104 to be maintained even as a vector 506 moves in and out of the 3D virtual model 102 along a linear path perpendicular to the front and back side planes. For example, if the probe 506 representing a vector moves a certain distance X into or out from the 3D virtual model 102, thus moving the focal point a distance x along the vector, then the modeling computer 106 automatically adjusts the range clipping parameters, thus moving both the front and back side planes defining the clipped slice in a proportional in order to maintain the same defined depth or width. In other words, the clipped slice 502 illustrated in FIG. 5A and the clipped slice 504 illustrated in FIG. 5B both have the same depth and are both sliced perpendicular to one another along the same linear path, although are sliced along different locations along that same liner path.

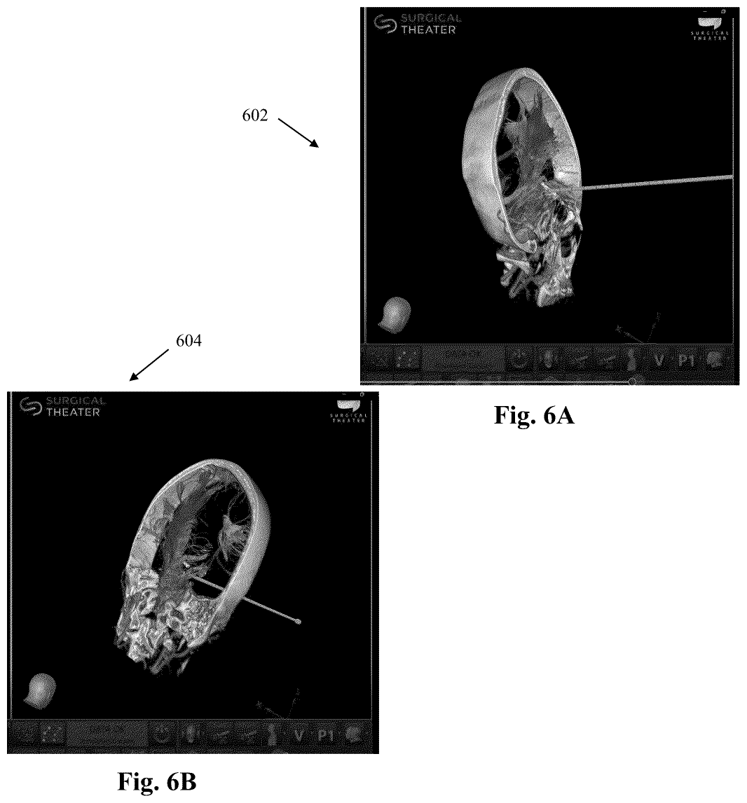

In one example, as illustrated in FIGS. 6a and 6b, the modeling computer 106 may enable range clipping and the desired depth of the clipped portion 104 to be maintained even as the probe, or other tool defining the vector, moves or changes positions, For example, if the probe tilts to a different angle, the modeling computer 106 automatically adjusts a clipped portion 602 from a first position to a clipped portion 604 in a second posit to reflect the new vector represented by the new position of the probe. Similarly, if a user's perspective of view moves, the modeling computer 106 similarly adjusts the clipped portion to reflect the new vector represented by the new view of the user. In other words, the vector and in turn the focal point that defines how the clipped portion 104 is represented to the user is determined based on tracking information received representative of movement an object or a user's view.

Referring back to FIG. 1, in order to adjust the clipped portion based on tracked movement, the modeling computer 106 may receive tracking data 120 from a navigation system 118. Based on the tracking data 120, the modeling computer 106 is able to adjust the vector so that it corresponds to a movement of a tool or movement of a user, for example.

Once the modeling computer 106 generates the clipped portion 104, the modeling computer 106 may communicate the 3D virtual model 102 along with the clipped portion 104 to a display. In one example, the display includes a HMD 116. In one example, the system 100 further enables augmenting and synchronizing the 3D virtual model 102 and the clipped portion 104 with a physical model 110. In one example, the modeling computer 106 enables the projection of the clipped portion 104 over top of a physical model 110.

The modeling computer 106 further enables the user 108 to view the augmented realty view of the physical model 110 from any perspective of the physical model 110. In other words, the user 108 may walk around the physical model 110 or move a probe around in any angle or direction and view a corresponding clipped portion 104 of the 3D virtual model 102 from any side, angle, or perspective, and to have a synchronized corresponding view of the clipped portion 104 of the 3D virtual model 102 overlayed on top of the physical model 110 in order to form the augmented realty view.

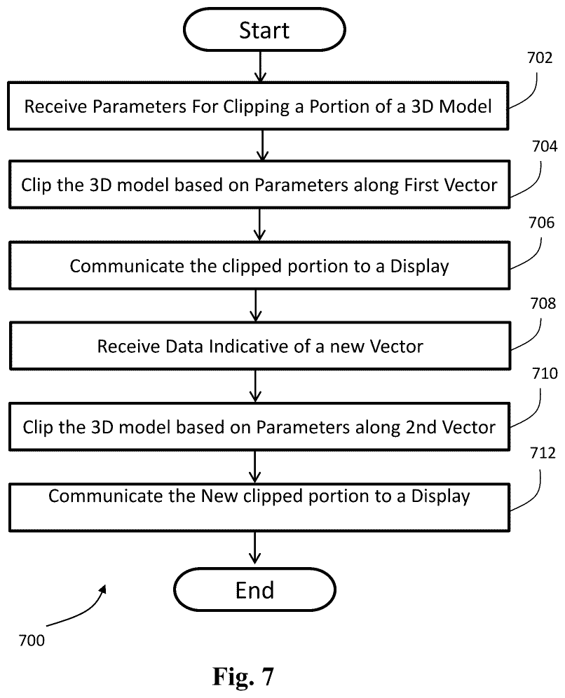

FIG. 7 illustrates an example synchronized 3D modeling clipping method. At 702, the modeling computer 106 receives parameters for clipping a portion of a 3D model. At the 704, the modeling computer 106 clips the 3D model according to the received parameters along a first vector. At 706, the modeling computer communicates the clipped portion of the 3D model to a display. At 708, the modeling computer 106 receives data indicative of a second vector. At 710, the modeling computer 106 clips the 3D model according to the received parameters along the second vector. At 712, modeling computer communicates the new clipped portion of the 3D model to a display.

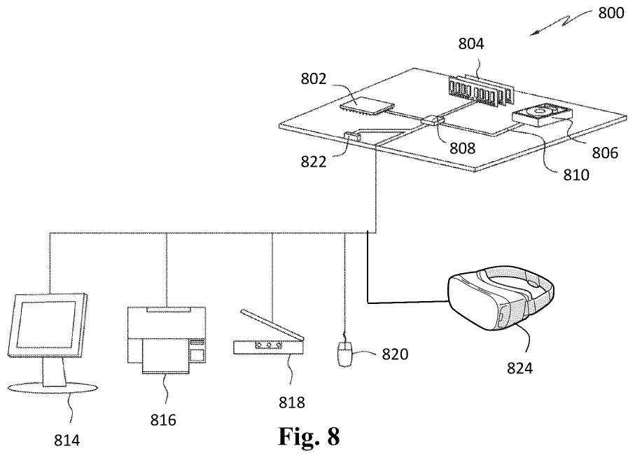

FIG. 8 is a schematic diagram of an example computer for implementing the modeling computer 106 of FIG. 1. The example computer 800 is intended to represent various forms of digital computers, including laptops, desktops, handheld computers, tablet computers, smartphones, servers, and other similar types of computing devices. Computer 800 includes a processor 802, memory 804, a storage device 806, and a communication port 822, operably connected by an interface 808 via a bus 810.

Processor 802 processes instructions, via memory 804, for execution within computer 800. In an example embodiment, multiple processors along with multiple memories may be used.

Memory 804 may be volatile memory or non-volatile memory. Memory 804 may be a computer-readable medium, such as a magnetic disk or optical disk. Storage device 806 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 804 or storage device 806.

Computer 800 can be coupled to one or more input and output devices such as a display 814, a printer 816, a scanner 818, a mouse 820, and a HMD 824.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C#, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A method for synchronized clipping of a 3D model, comprising the steps of:
a modeling computer receiving data indicative of a frame of reference with respect to a vector;
the modeling computer generating data indicative of first parameters, based on the frame of reference, for clipping a 3D model of a patient anatomy along the vector;
the modeling computer clipping the 3D model based on the first parameters, thereby generating a first clipped portion;
the modeling computer communicating the first clipped portion to a display;
the modeling computer receiving data indicative of movement within the frame of reference;
the modeling computer automatically regenerating second parameters, based on and in sync with the data indicative of the movement, for clipping the 3D model;
the modeling computer clipping the 3D model based on the second parameters, thereby generating a second clipped portion; and
the modeling computer communicating the second clipped portion to the display.

2. The method of claim 1, wherein the vector is representative of a virtual medical instrument and wherein the frame of reference comprises at least one of a position, angle, and focal point of the medical instrument.

3. The method of claim 2, wherein the vector representative of the medical instrument corresponds to a physical medical instrument, wherein the method further comprises the steps of the modeling computer receiving tracking coordinates representative of the physical position of the physical medical instrument, and the modeling computer automatically generating the second parameters based on the movement of the physical medical instrument.

4. The method of claim 1, wherein the vector is representative of the position and angle of view of a physical user, wherein the method further comprises the steps of the modeling computer receiving tracking coordinates representative of the physical position and angle of view of the physical user, and the modeling computer automatically generating the second parameters based on the movement of the physical user.

5. The method of claim 1, wherein the method further comprises the steps of the modeling computer receiving data representative of customizable adjustments to the first parameters and the second parameters.

6. The method of claim 1, wherein the customizable adjustments comprises data indicative of a thickness of a clipped portion, including data indicative of the position along the vector of at least one of the front and the back of the clipped portion.

7. The method of claim 6, wherein the method further comprises the steps of the modeling computer automatically adjusting and maintaining the thickness of the clipped portion during movement within the frame of reference.

8. The method of claim 1, wherein the method further comprises the steps of the modeling computer receiving tracking data indicative of a location and tracked movement with respect to a physical patient, and the modeling computer communicating a clipped portion to a display such that the clipped portion is augmented and synchronized with the physical patient based on the tracking data.

9. A synchronized clipping modeling computer, comprising:
a first module for receiving data indicative of a frame of reference with respect to a vector;
a second module for generating data indicative of first parameters, based on the frame of reference, for clipping a 3D model of a patient anatomy along the vector;
a third module for clipping the 3D model based on the first parameters, thereby generating a first clipped portion; and
a fourth module for communicating the first clipped portion to a display;

10. The synchronized clipping modeling computer of claim 9, further comprising:
a fifth module for receiving data indicative of movement within the frame of reference;
wherein the second module is further configured for automatically regenerating second parameters, based on and in sync with the data indicative of the movement, for clipping the 3D model;
wherein the third module is further configured for clipping the 3D model based on the second parameters, thereby generating a second clipped portion; and
wherein the fourth module is further configured for communicating the second clipped portion to the display.

11. The synchronized clipping modeling computer of claim 9, wherein the vector is representative of a virtual medical instrument and wherein the frame of reference comprises at least one of a position, angle, and focal point of the medical instrument.

12. The synchronized clipping modeling computer of claim 11, wherein the vector representative of the medical instrument corresponds to a physical medical instrument, wherein the synchronized clipping modeling computer is configured to receive tracking coordinates representative of the physical position of the physical medical instrument, and wherein synchronized clipping modeling computer is configured to automatically generate the second parameters based on the movement of the physical medical instrument.

13. The method of claim 9, wherein the vector is representative of the position and angle of view of a physical user, wherein the synchronized clipping modeling computer is configured to receive tracking coordinates representative of the physical position and angle of view of the physical user, and wherein the synchronized clipping modeling computer is configured to automatically generate the second parameters based on the movement of the physical user.

14. The method of claim 9, wherein the synchronized clipping modeling computer is configured to receive data representative of customizable adjustments to the first parameters and the second parameters.

15. The method of claim 9, wherein the customizable adjustments comprises data indicative of a thickness of a clipped portion, including data indicative of the position along the vector of at least one of the front and the back of the clipped portion.

16. The method of claim 15, wherein the synchronized clipping modeling computer is configured to automatically adjust and maintain the thickness of the clipped portion during movement within the frame of reference.

17. The method of claim 9, wherein the synchronized clipping modeling computer is configured to receive tracking data indicative of a location and tracked movement with respect to a physical patient, and wherein the synchronized clipping modeling computer is configured to communicate a clipped portion to a display such that the clipped portion is augmented and synchronized with the physical patient based on the tracking data.

18. A system, comprising:
a first module for receiving data indicative of a frame of reference with respect to a vector;
a second module for generating data indicative of first parameters, based on the frame of reference, for clipping a 3D model of a patient anatomy along the vector;
a third module for clipping the 3D model based on the first parameters, thereby generating a first clipped portion;
a fourth module for communicating the first clipped portion to a display; and
a fifth module for receiving data indicative of movement within the frame of reference;
wherein the second module is further configured for automatically regenerating second parameters, based on and in sync with the data indicative of the movement, for clipping the 3D model;
wherein the third module is further configured for clipping the 3D model based on the second parameters, thereby generating a second clipped portion; and
wherein the fourth module is further configured for communicating the second clipped portion to the display.
